(19) United States
(12) Patent Application Publication
(10) Pub. No.: US 2007/0298109 A1
(43) Pub. Date: Dec. 27, 2007

(54) Nano-scale devices

(75) Inventors: Larry J. Kricka, Devon, PA (US); Jason Y. Park, Philadelphia, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: The Trustees Of The University Of Pennsylvania, Philadephia, PA (US)

(21) Appl. No.: 11/176,407

(22) Filed: Jul. 7, 2005

(51) Int. Cl. A61K 9/14; B29B 9/00

(52) U.S. Cl. 424/489; 264/5; 977/906; 425/6

(57) ABSTRACT

Nano-scale devices with filter zones that define the size of the resulting nanoparticles. The filter zones may have nanotubes with diameters that are the maximum dimension of the resulting particles or the filter zones may have filtering particles arranged in a predetermined array with interstices defining the maximum dimension of the resulting nanoparticles.

### FIELD OF THE INVENTION

This invention relates to nano-scale devices having filter zones that produce nanoparticles of a maximum predetermined size. Also included are methods of making nanoparticles using the devices of the present invention.

### BACKGROUND OF THE INVENTION

Micromachining technology has been used to manufacture microfabricated reactors. Recently, these reactors have been used to perform synthetic chemical and biochemical analyses. Reaction conditions are more easily controlled on a smaller scale than in larger, bulkier reactor systems. The large reactor systems are often inefficient and wasteful as they may also require reagent amounts that exceed what is available. Microfabricated reactors also allow more rapid diffusion and mixing, superior heat transfer, and a reproducible and defined reaction environment that leads to higher product yields. The use of such reactors has already been recognized for microparticle production and has been applied to nanoparticles. Microreactors of different types are commercially available.

Microreactors, or microchannel reactors, have channels etched into a substrate such as glass. In some reactors, silicon is bonded to the glass. The reactors typically have residence zones that act as reaction channels and microfluidic ports acting as inlets and outlets. The reactors can be filled by using syringe pumps, which can provide continuous or on demand flow rates. There are other reactors with microfabricated filters used for analysis and for fluid handling, conducting polynucleotide amplification reactions, and analyte detection. Many current reactors, however, typically lack means of producing uniform particles.

In some systems, gold particles are used as “seeds” for the generation of larger particles. These particles are combined with reagent solutions in the reactor system. The diameters of the resulting particles vary widely, however. Other nanoparticle production methods using, for example, gold colloids, quantum dots, and nanocrystals and nanoshells, provide relatively poor yields of polydisperse product. Using these nanostructures as imaging or therapeutic agents requires a higher degree of control of the reaction than has been previously utilized so that high yields of monodisperse products can be obtained.

Nanoparticulates, solid nanoparticles, polymeric nanoparticles, and polymeric self-assemblies and nanosuspensions can also be used as drug delivery systems for substances with poor solubility and absorption characteristics. A basic problem for drug delivery is poor solubility of the macroscale compounds. This poor solubility leads to unpredictable absorption and low bioavailability. Indeed, poor solubility leads to limitations in the routes of administration. Nanoparticles have improved saturation solubility and dissolution velocity. Examples of compounds include high molecular weight substances, peptides, proteins, oligonucleotides and plasmids. Nanoscale particles have been produced by milling, high pressure homogenization, emulsification, precipitation, or polymerization at the nanoscale.

Partial solutions that are currently used to enhance solubility are mixed micelles (e.g., Valium MM® for i.v. injection), non-specific or specific admixtures (e.g., use of polyethyleneglycol or cyclodextrins), and solvent mixtures (e.g., ethanol-water). These formulations, while helpful, have had limited commercial success.

Another approach to improve solubility of drugs has been to micronize by milling. Simple micronization increases the dissolution rate of the drug by dramatically increasing surface area. However, this dissolution does not improve the saturation solubility. An evolution of micronization was the production of nanosized drug particles. This was initially performed by precipitation of the drug from a solvent. During the precipitation of the drug from the solvent, the crystals would be exposed to surfactants that would limit the formation of larger particles. However, this precipitation technique requires solubility in a given solvent as well as good mixibility with the precipating non-solvent. Nanoparticulates can also be produced by a milling process with exposure to a surfactant solution (e.g., NanoCrystals® by Elan).

The second generation drug nanoparticles are produced by high pressure homogenization leading to nanosuspensions. Nanosuspensions are produced by high pressure cavitation forces created in homogenizers. These nanosuspensions may be used as drug formulations. Examples of nanosuspension drugs are paclitaxel (300 nm particle size), clofaxemine (600 nm particle size), and RMKP22 (540 nm particle size). Drug formulations may be modified by altering the surface of the nanoparticles with surfactants or polymers. Two major approaches to nanoparticle preparation of drugs is precipitation leading to hydrosols or alternatively, ultra-fine milling.

Nanosuspensions and nanoparticles have several benefits: (1) Increased drug solubility leads to increased drug amount in dosage without harsh vehicles; (2) small particle size leads to faster drug dissolution and increased rate and extent of absorption. The general process of manufacturing nanosuspensions involves two phases: (1) creation of a crystal nuclei; (2) growth of the crystal. The production of a stable suspension of small particle size requires a high nucleation rate coupled with a low growth rate. High-supersaturation conditions are used for rapid nucleation by rapidly mixing water-miscible organic solvent containing the drug with non-solvent water. This rapid dilution leads to spontaneous nucleation. There are some solid-particulate-nanosuspension-based formulations currently on the market. However, many of these formulations as well as reactors used to make them do not produce uniform particles in a carefully controlled manner. As such, there is a need for efficient means for nanosizing drugs to improve solubility and absorption characteristics of those drugs having poor solubility.

### SUMMARY OF THE INVENTION

The present invention provides devices for the synthesis of nanoparticles. The devices are useful in diagnostic testing, tracer studies, and imaging. Some embodiments are useful in making nano-formulations of drugs at the synthetic stage or formulation stage. The devices comprise at least one inlet port and at least one exit port. There is also a flow channel and reaction channel in fluid communication with the inlet and exit ports. A filter zone within the reaction channel comprises filtering particles arranged in a predetermined array. The predetermined array may define a lattice structure in some embodiments. The interstices among the particles define a maximum dimension of the resulting nanoparticles. In many embodiments, the filter zone with the reaction channel may comprise nanotubes arranged in parallel with the flow of fluid through said filter zone. The diameters of the nanotubes define a maximum dimension of the nanoparticles. There are also embodiments wherein the filter zone within the reaction channel comprises nanopores etched into a substrate.

Although there are a number of interpretations of the preferred or optimal size range of nanoparticles, this invention provides a means to adjust the maximum nanoparticle dimension size. As such, there are provided embodiments where the devices synthesize particles with a maximum dimension that is less than about 1 μm. In embodiments that may be preferred, the maximum dimension is less than about 400 nm for drug formulations. Some embodiments are fabricated in silicon, glass, quartz, or plastic. The device may have three inlet ports. Some embodiment may also be capped with glass. The particles made by some embodiments may be gold colloid particles, quantum dots, nanocrystals, nanobeads, nanoshells, polymer particles, or drug particle formulations.

The present invention also provides methods of making nanoparticles having a maximum dimension less than about 1 μm comprising using an embodiment of the devices disclosed herein. The method comprises adding reactants through said inlet ports and cycling the reactants through the reaction channel to provide nanoparticles. In some embodiments, the reactants may be added sequentially or simultaneously. The device itself may be heated, cooled, or exposed to radiation during the process. The reactants may comprise among other things, ethanol, ammonium hydroxide, or tetraethyl orthosilicate.

Also provided are filters for nanoscale flow systems comprising filtering particles arranged in a predetermined array. The array may define a lattice with the filtering particle interstices defining a maximum dimension of the nanoparticles produced by the flow system in some embodiments. Other embodiments of the filters may comprise nanotubes arranged in parallel with the flow of fluid through said system. Nanopores etched into a substrate may also be used as the filter in some embodiments. The diameters of the nanotubes and nanopores define a maximum dimension of nanoparticles exiting the system.

The present invention also provides methods of making drug formulations using the devices described herein with a method comprising adding a drug substance and surface modifier through the inlet ports and cycling the drug substance and surface modifier through the reaction channel to reduce the particle size of said drug substance to an effective particle size for drug delivery.

### BRIEF DESCRIPTION OF THE DRAWINGS

FIG. 1 represents a exemplary nanoscale device design for producing nanoparticles in accordance with the present disclosure.

FIG. 2 depicts two embodiments of the present invention where the filter is an assembly of nanotubes or nanopores etched into a substrate.

### DETAILED DESCRIPTION OF ILLUSTRATIVE EMBODIMENTS

Embodiments of the nano-scale devices of the present invention are an advance over currently existing methods for producing nanoparticles. Such nanoparticles may include, but are not limited to, colloid particles, quantum dots, nanocrystals, nanobeads, nanoshells, polymer particles, or drug particle formulations. Embodiments of the present invention achieve an exemplary level of control of reaction conditions in chemical reactions designed to produce microstructures via growth processes. This level of control is at least partially due to the filter zone present in embodiments of the present invention.

In embodiments of the present invention that may be preferred, there are devices having nanoscale channels that may be microfabricated from substrates such as silicon, glass, quartz, or plastic. In some embodiments, the device 100 may be capped with glass using anodic bonding. The flow channels 110 and reaction channels 120 may be designed and fabricated from solid substrates using established micromachining methods such as photolithography, etching and deposition techniques, laser machining, LIGA processing and plastic molding. In some embodiments, the devices 100 may be constructed by fabricating flow channels 110 and one or more reaction channels 120 into the surface of the substrate, and then adhering or clamping a cover over the surface. Along with the substrate, the cover may also comprise a material such as silicon, polysilicon, silica, glass, gallium arsenide, polyimide, silicon nitride, and silicon dioxide. The cover and substrate alternatively may comprise a plastic material such as an acrylic, polycarbonate polystyrene, or polyethylene. In other embodiments, the cover or substrate may comprise a transparent or non-transparent material. In such embodiments, the covers may be necessary for UV transparency for UV-initiated polymerization reactions.

The nano-scale devices comprise at least one entry port 130 in communication with a flow channel 110. Some embodiments may comprise 2, 3, or more entry ports 130. There is also at least one exit port 140 for resulting nanoparticles 210. Reactants for making nanoparticles 210 may vary depending on the desired particles and the needs of the system. Exemplary reactants include, but are not limited to, ethanol (EtOH), ammonium hydroxide (NH₄OH), or tetraethyl orthosilicate (TEOS). Other reactants include silica; gold; silver; bismuth chloride and chromium dichloride; sodium sulfide or hydrogen sulfide with lead nitrate or lead acetate; Me₂EtAlH₃ with titanium isopropoxide (catalyst); molybdenum chloride with NaBEt₃H; trimethylindium with phosphine; ferrous chloride with potassium ferricyanide; H₂PtCl₆ with sodium borohydride; cobalt acetate with trioctylphosphine with 1,2-dodecanediol; selenium in trioctyl phosphine with cadmium acetate in trioctyl phosphine oxide; titanium chloride; dimethylcadmium, selenium, tributylphosphine and trioctyl phosphine oxide; nanoshells; polymer, such as polymer in acetone with aqueous pluronic F68®; copolymer, such as a UV-initiated copolymerization of a mixture of acrylic acid (5%) with tripropylene glycol diacrylate in 2% sodium dodecyl sulfate with 4 wt % 1-hydroxycyclohexyl phenyl ketone (photo-initiator).

The devices also comprise at least one reaction channel comprising filter zones. There may be a number of reaction channels in a given device. In some embodiments, there are two reaction channels with one channel used for short periods of incubation and a second channel for longer periods of incubation. In one of the reaction channels are filter zones that control and limit the size of the growing nanoparticles by virtue of the interstices of the filtering particles 160. The filtering particles 160 are packed into the filter zone 150 in a predetermined array, which, in some embodiments, may define a lattice. Cycling of the reaction mixture (i.e., a back and forth flow) through the reaction channel constantly sieves the reaction mixture. This restricts a dimension of the resulting particles to the size of the interstices in the filter particle array 170. At the end of the process, larger sized particles will be retained while particles with diameters equal to or less than the size of the interstices will be harvestable.

The filter functionality may be achieved by packing the reaction channel with filtering particles 160 or nanotubes 180 to provide the filter zone 150. There are also embodiments made by nanopores 190 etched into the substrate 200. The filter zones 150 may comprise filtering particles 160 arranged in a predetermined array 170. This array 170 may define a lattice structure or considered a membrane with nanopores 190 or a bed of beads. The interstices among the filtering particles define a maximum dimension of the resulting nanoparticles 210. In other embodiments, the diameter of the nanotubes 180 or the nanopores 190 define the maximum diameter of the nanoparticles 210.

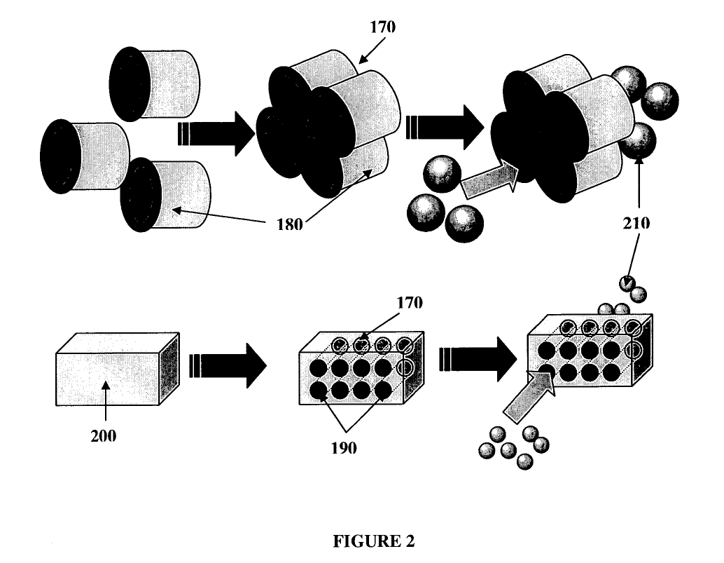

The diameter of the nanotubes 180 may define a maximum particle dimension. As seen in FIG. 2, the filter array 170 may be made of an assembly of nanotubes 180 or nanopores 190 etched into a substrate 200. The nanotubes 180 may comprise metal, carbon, silicon, composites, protein, DNA, polymers, and coblock polymers. The nanopores 190 may be made by etching holes or channels into substrates 200 such as silicon, metal, carbon, composites, proteins, polymers, and coblock polymers.

As some resulting nanoparticles may comprise polymer, it is foreseeable that these particles have some degree of deformability. In some embodiments, the nanoparticles may comprise clay, composites, metals, or core-shell configurations. Deformability of nanoparticles have been shown using other methods such as ion irradiation with nanosized particles composed of clay, silica, ZnS, and SiO₂/Au core-shell colloids. Aluminum and copper nanoparticles have also been deformed by other methods. The resulting deformable nanoparticles may have a maximum dimension slightly greater than defined by the interstices, nanotube diameters, or nanopore diameters in the filter zones.

Therefore, there are embodiments where either the interstices among the filtering particles 160, the nanotube 180 diameters, or the nonopore 190 diameters define the maximum diameters of the resulting nanoparticles 210. The nanotubes 180 form conduits along which the reaction mixture flows within the reaction channel. In some embodiments, the filter zones 150 comprise nanotubes 180 with lengths greater than the widths of the reaction channel and the nanotubes 180 have diameters defining a maximum dimension of the resulting nanoparticles 210. The nanotubes 180 are longer than the width of the reactor channel so that they will pack parallel to the flow of the fluid through the filter zone 150.

The filters retain the nanoparticles 210 greater than the maximum dimension defined by either the interstices of the filtering particles or the nanotube 180 diameters, thus allowing those nanoparticles 210 of preferred dimension to flow through to the exit port 130. In some embodiments, that may be preferable, the preferred maximum dimension may be about 5 nm where a filter zone 150 is tightly packed longitudinally with 5 nm diameter nanotubes 180. The maximum dimension may be up to 100 nm in some embodiments and all combinations or subcombinations therein. The dimension may be within the range of about 2 nm to about 10 nm in other embodiments that may be preferred.

A range of processing capabilities are possible in the embodiments of the present invention. These include, but are not limited to, sequential or simultaneous addition of reagents, mixing, separation, size-dependent filtration, heating, cooling, exposure to radiation, on-board sensors, direct visualization or UV-VIS monitoring of reactions and, integration with other nano-scale devices. Scale of production can be increased by assembling groups of reactors or by scaling up of on-chip features such as chamber sizes.

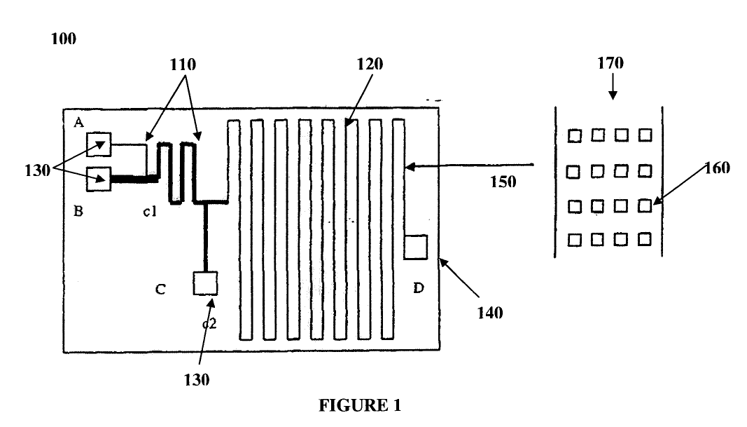

Optimization of some embodiments of the present invention will rely on iterative techniques based on the diversity of embodiments that can be fabricated on a single wafer. An exemplary design for an embodiment of the present invention is shown in FIG. 1. The entry ports 130 are designated A, B, and C, with the exit port 140 being D. In some embodiments, for example, EtOH can be added to A; NH₄OH can be added to B; and TEOS can be added to C. At the end of the reaction process, the resulting nanoparticle exits through port D. The flow rates into the channels linked to A and B are 25:1. Incubation coils c1 and c2 provide for short and long periods of incubation. The chip or wafer containing the nano-scale device may be heated or cooled, and fluid flow can be produced by an external pump attached to the entry ports 130.

The present invention also provides methods for making nanoparticles of prescribed diameter using the devices described above. The methods comprise adding reagents to the devices and cycling said reaction mixture through the flow and reaction channels to provide nanoparticles. The reactants may be added sequentially or simultaneously depending on the number of inlet port and method chosen by the operator. The devices may be heated, cooled, or exposed to radiation during the process.

There are also embodiments of the present invention that may be used to produce drug formulations. A drug having poor water solubility may be dissolved in a suitable solvent and then recrystallized in the nano-confined nanofilter environment such as those disclosed herein. As indicated in the U.S. Pat. No. 5,145,684 to Liversidge, incorporated herein by reference in its entirety, the recrystallized drug can by stabilized as a nanoparticulate by the addition of surface modifiers. Such surface modifiers may be added to nanoparticulates using the devices of the present invention.

To that end, there are methods of making drug formulations using the devices described above with a method comprising adding a drug substance and surface modifier through said inlet ports and cycling the drug substance and surface modifier through the reaction channel to provide drug formulations with a particle size of less than about 1 μm. In some preferred embodiments, a particle size range less than about 400 nm may be preferred. The drug substance may be any medicament or pharmaceutically acceptable substance known or suspected to promote recovery from ailment. Steroids such as danazol, Steroid A, or an antiviral agent are examples of drug substances that may be used in some embodiments. The surface modifier may comprise gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene caster oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, and polyvinylpyrrolidone. The surface modifier may also comprise polyvinylpyrrolidone, an ethylene oxide-propylene oxide block copolymer, lecithin, an alkyl aryl polyether sulfonate, gum acacia, sodium dodecylsulfate, and a dioctylester of sodium sulfosuccinic acid.

A nanofilter array as previously described for the fabrication of nanoparticles may also be used for the fabrication of nanosuspensions and nanoparticles with the intent of drug utility. An illustrative species of drug substances that should be useful are shown in U.S. Pat. No. 5,145,684 to Liversidge. They include: 17-α-pregno-2,4-dien-20-yno-[2,3-d]-isoxazol-17-ol (Danazol); 5α,17α,-1′-(methylsulfonyl)-1′H-pregn-20-yno[3,2-c]-pyrazol-17-ol (Steroid A); piposulfam; piposulfan; camptothecin; or ethyl-3,5-diacetoamido-2,4,6-triiodobenzoate.

### EXAMPLE 1

### Methods for Producing Nanoparticles and Nanoshells

Gold particles are produced by reacting a boiling solution of ˜5.0 mM HAuCl₄ in water with a reducing agent such as 0.5% sodium citrate with continuous stirring, or ascorbic acid, phosphorus, or borohydride.

Silver particles are produced by reacting a boiling solution of 5.0 mM silver nitrate in water with a reducing agent such as 1% sodium citrate or ascorbic acid, phosphorus, borohydride, or potassium bitartrate.

Nanoshells are made by: Step 1—Grow silica nanoparticles of diameter 20 nm diameter by reducing tetraethyl 0.1 M-0.5 M orthosilicate with 0.5 M-3 M ammonium hydroxide in EtOH. The size of particle are determined by water, base, and TEOS concentration and are viewed using a SEM microscope. Size uniformity should be SD 4%-10%.

Step 2—Attach seed colloid (e.g., gold or silver, 1-2 nm diameter) via chemical linkages to nanoparticle surface. Aminate the silica surface with aminopropyltriethoxysilane in EtOH. Attach more gold colloid of 1 nm-3 nm diameter.

Step 3—Grow additional metal onto surface via chemical reduction of chlorauric acid in K₂CO₃ and formaldehyde. Assess formation using UV-VIS spectrophotometry and size particles using a SEM and TEM.

What is claimed:

1. A device for the synthesis of nanoparticles comprising:
at least one inlet port and at least one exit port;
a flow channel and reaction channel in fluid communication with said ports; and
a filter zone within said reaction channel, with said filter zone comprising filtering particles arranged in a predetermined array.

2. The device of claim 1 wherein said array defines a lattice.

3. The device of claim 1 wherein the interstices among said filtering particles define a maximum dimension of said nanoparticles.

4. The device of claim 3 wherein said dimension is less than about 1 μm.

5. The device of claim 3 wherein said dimension is less than about 400 nm.

6. The device of claim 1 comprising three inlet ports.

7. The device of claim 1 fabricated in silicon, glass, quartz, or plastic.

8. The device of claim 1 capped with glass.

9. The device of claim 1 wherein said nanoparticles are gold colloid particles, quantum dots, nanocrystals, nanobeads, nanoshells, polymer particles, or drug particle formulations.

10. The device of claim 1 wherein said nanoparticles are deformable.

11. The device of claim 1 wherein said nanoparticles comprise clay, composites, metals, or core-shell configurations.

12. A device for the synthesis of nanoparticles having a maximum dimension less than about 1 μm comprising:
at least one inlet port and at least one exit port;
a flow channel and reaction channel in communication with said ports; and
a filter zone within said reaction channel, with said filter zone comprising filtering particles arranged in a predetermined array and the interstices among said filtering particles define said dimension.

13. The device of claim 12 wherein said array defines a lattice.

14. The device of claim 12 wherein said maximum dimension is less than about 400 nm.

15. The device of claim 12 comprising three inlet ports.

16. The device of claim 12 fabricated in silicon, glass, quartz, or plastic.

17. The device of claim 12 capped with glass.

18. The device of claim 12 wherein said nanoparticles are gold colloid particles, quantum dots, nanocrystals, nanobeads, nanoshells, polymer particles, or drug particle formulations.

19. The device of claim 12 wherein said nanoparticles are deformable.

20. The device of claim 12 wherein said nanoparticles comprise clay, composites, metals, or core-shell configurations.

21. A device for the synthesis of nanoparticles comprising:
at least one inlet port and at least one exit port;
a flow channel and reaction channel in communication with said ports; and
a filter zone within said reaction channel, with said filter zone comprising nanotubes arranged in parallel with the flow of fluid through said filter zone.

22. The device of claim 21 wherein the diameters of said nanotubes define a maximum dimension of said nanoparticles.

23. The device of claim 22 wherein said dimension is less than about 1 μm.

24. The device of claim 23 wherein said dimension is less than about 400 nm.

25. The device of claim 21 comprising three inlet ports.

26. The device of claim 21 fabricated in silicon, glass, quartz, or plastic.

27. The device of claim 21 capped with glass.

28. The device of claim 21 wherein said nanoparticles are gold colloid particles, quantum dots, nanocrystals, nanobeads, nanoshells, polymer particles, or drug particle formulations.

29. The device of claim 21 wherein said nanoparticles are deformable.

30. The device of claim 21 wherein said nanoparticles comprise clay, composites, metals, or core-shell configurations.

31. The device of claim 21 wherein said nanotubes comprise metal, carbon, silicon, composites, protein, DNA, polymers, or coblock polymers.

32. A device for the synthesis of nanoparticles having a maximum dimension less than about 1 μm comprising:
at least one inlet port and at least one exit port;
a flow channel and reaction channel in communication with said ports; and
a filter zone within said reaction channel, with said filter zone comprising nanotubes arranged in parallel with the flow of fluid through said filter zone with the diameters of said nanotubes defining said maximum dimension.

33. The device of claim 32 wherein said dimension is less than about 400 nm.

34. The device of claim 32 comprising three inlet ports.

35. The device of claim 32 fabricated in silicon, glass, quartz, or plastic.

36. The device of claim 32 capped with glass.

37. The device of claim 32 wherein said nanoparticles are gold colloid particles, quantum dots, nanocrystals, nanobeads, nanoshells, polymer particles, or drug particle formulations.

38. The device of claim 32 wherein said nanoparticles are deformable.

39. The device of claim 32 wherein said nanoparticles comprise clay, composites, metals, or core-shell configurations.

40. The device of claim 32 wherein said nanotubes comprise metal, carbon, silicon, composites, protein, DNA, polymers, or coblock polymers.

41. A device for the synthesis of nanoparticles comprising:
at least one inlet port and at least one exit port;
a flow channel and reaction channel in fluid communication with said ports; and
a filter zone within said reaction channel, with said filter zone comprising nanopores etched into a substrate.

42. The device of claim 41 wherein the diameters of said nanopores define a maximum dimension of said nanoparticles.

43. The device of claim 42 wherein said dimension is less than about 1 μm.

44. The device of claim 43 wherein said dimension is less than about 400 nm.

45. The device of claim 41 comprising three inlet ports.

46. The device of claim 41 fabricated in silicon, glass, quartz, or plastic.

47. The device of claim 41 capped with glass.

48. The device of claim 41 wherein said nanoparticles are gold colloid particles, quantum dots, nanocrystals, nanobeads, nanoshells, polymer particles, or drug particle formulations.

49. The device of claim 41 wherein said nanoparticles are deformable.

50. The device of claim 41 wherein said nanoparticles comprise clay, composites, metals, or core-shell configurations.

51. The device of claim 41 wherein said substrate is silicon, metal, carbon, composites, proteins, polymers, or coblock polymers.

52. A method of making nanoparticles having a maximum dimension less than about 1 μm comprising using a device comprising:
at least one inlet port for the addition of reactants and at least one exit port for said nanoparticles;
a flow channel and reaction channel in communication with said ports; and
a filter zone within said reaction channel, with said filter zone comprising filtering particles arranged in a predetermined array, wherein the interstices among said filtering particles define said dimension;
said method comprising:
adding reactants through said inlet ports; and
cycling said reactants through said reaction channel to provide said nanoparticles.

53. The method of claim 52 wherein said array defines a lattice.

54. The method of claim 52 wherein said dimension is less than about 400 nm.

55. The method of claim 52 wherein said reactants are added sequentially or simultaneously.

56. The method of claim 52 further comprising heating, cooling, or exposing the device to radiation.

57. The method of claim 52 wherein said reactants comprise ethanol, ammonium hydroxide, tetraethyl orthosilicate, silica, gold, or silver.

58. The method of claim 52 wherein said reactants comprise bismuth chloride, chromium dichloride, sodium sulfide, hydrogen sulfide, lead nitrate, lead acetate, Me₂EtAlH₃, titanium isopropoxide, molybdenum chloride, NaBEt₃H, trimethylindium, phosphine, ferrous chloride, potassium ferricyanide, H₂PtCl₆, sodium borohydride, cobalt acetate, trioctylphosphine, 1,2-dodecanediol, selenium in trioctyl phosphine, cadmium acetate in trioctyl phosphine oxide, titanium chloride, dimethylcadmium, selenium, tributylphosphine and trioctyl phosphine oxide, or nanoshells.

59. The method of claim 52 wherein said reactants comprise polymer or copolymer.

60. The method of claim 59 wherein said polymer or copolymer comprise poly(ε-caprolactone), poly(D,L-lactide), or poly(tripropylene glycol diacrylate/acrylic acid).

61. The method of claim 52 wherein said nanoparticles are gold colloid particles, quantum dots, nanocrystals, nanobeads, nanoshells, polymer particles, or drug particle formulations.

62. The method of claim 52 wherein said nanoparticles are deformable.

63. The method of claim 52 wherein said nanoparticles comprise clay, composites, metals, or core-shell configurations.

64. A method of making particles having a maximum dimension less than about 1 μm using a device comprising:
at least one inlet port for the addition of reactants and at least one exit port for said nanoparticles;
a flow channel and reaction channel in communication with said ports; and
a filter zone within said reaction channel, with said filter zone comprising nanotubes arranged in parallel with the flow of fluid through said filter zone with the diameters of said nanotubes defining said maximum dimension;
said method comprising:
adding reactants through said inlet ports; and
cycling said reactants through said reaction channel to provide said resulting particles.

65. The method of claim 64 wherein said maximum dimension is less than about 400 nm.

66. The method of claim 64 wherein said reactants are added sequentially or simultaneously.

67. The method of claim 64 further comprising heating, cooling, or exposing the device to radiation.

68. The method of claim 64 wherein said reactants comprise ethanol, ammonium hydroxide, tetraethyl orthosilicate, silica, gold, or silver.

69. The method of claim 64 wherein said reactants comprise bismuth chloride, chromium dichloride, sodium sulfide, hydrogen sulfide, lead nitrate, lead acetate, Me₂EtAlH₃, titanium isopropoxide, molybdenum chloride, NaBEt₃H, trimethylindium, phosphine, ferrous chloride, potassium ferricyanide, H₂PtCl₆, sodium borohydride, cobalt acetate, trioctylphosphine, 1,2-dodecanediol, selenium in trioctyl phosphine, cadmium acetate in trioctyl phosphine oxide, titanium chloride, dimethylcadmium, selenium, tributylphosphine and trioctyl phosphine oxide, or nanoshells.

70. The method of claim 64 wherein said reactants comprise polymer or copolymer.

71. The method of claim 70 wherein said polymer or copolymer comprise poly(ε-caprolactone), poly(D,L-lactide), or poly(tripropylene glycol diacrylate/acrylic acid).

72. The method of claim 64 wherein said nanotubes comprise metal, carbon, silicon, composites, protein, DNA, polymers, or coblock polymers.

73. The method of claim 64 wherein said nanoparticles are gold colloid particles, quantum dots, nanocrystals, nanobeads, nanoshells, polymer particles, or drug particle formulations.

74. The method of claim 64 wherein said nanoparticles are deformable.

75. The method of claim 64 wherein said nanoparticles comprise clay, composites, metals, or core-shell configurations.

76. A method of making nanoparticles having a maximum dimension less than about 1 μm comprising using a device comprising:
at least one inlet port for the addition of reactants and at least one exit port for said nanoparticles;
a flow channel and reaction channel in communication with said ports; and
a filter zone within said reaction channel, with said filter zone comprising nanopores etched into a substrate;
said method comprising:
adding reactants through said inlet ports; and
cycling said reactants through said reaction channel to provide said nanoparticles.

77. The method of claim 76 wherein the diameters of said nanopores define a maximum dimension of said nanoparticles.

78. The method of claim 77 wherein said dimension is less than about 400 nm.

79. The method of claim 76 wherein said reactants are added sequentially or simultaneously.

80. The method of claim 76 further comprising heating, cooling, or exposing the device to radiation.

81. The method of claim 76 wherein said reactants comprise ethanol, ammonium hydroxide, tetraethyl orthosilicate, silica, gold, or silver.

82. The method of claim 76 wherein said reactants comprise bismuth chloride, chromium dichloride, sodium sulfide, hydrogen sulfide, lead nitrate, lead acetate, Me₂EtAlH₃, titanium isopropoxide, molybdenum chloride, NaBEt₃H, trimethylindium, phosphine, ferrous chloride, potassium ferricyanide, H₂PtCl₆, sodium borohydride, cobalt acetate, trioctylphosphine, 1,2-dodecanediol, selenium in trioctyl phosphine, cadmium acetate in trioctyl phosphine oxide, titanium chloride, dimethylcadmium, selenium, tributylphosphine and trioctyl phosphine oxide, or nanoshells.

83. The method of claim 76 wherein said reactants comprise polymer or copolymer.

84. The method of claim 83 wherein said polymer or copolymer comprise poly(ε-caprolactone), poly(D,L-lactide), or poly(tripropylene glycol diacrylate/acrylic acid).

85. The method of claim 76 wherein said substrate is silicon, metal, carbon, composites, proteins, polymers, or coblock polymers.

86. The method of claim 76 wherein said nanoparticles are gold colloid particles, quantum dots, nanocrystals, nanobeads, nanoshells, polymer particles, or drug particle formulations.

87. The method of claim 76 wherein said nanoparticles are deformable.

88. The method of claim 76 wherein said nanoparticles comprise clay, composites, metals, or core-shell configurations.

89. A method of making drug formulations using a device comprising:
at least one inlet port and at least one exit port the for resulting drug formulations;
a flow channel and reaction channel in communication with said ports; and
a filter zone within said reaction channel, with said filter zone comprising filtering particles arranged in a predetermined array;
said method comprising:
adding a drug substance and surface modifier through said inlet ports; and
cycling said drug substance and surface modifier through said reaction channel to provide drug formulations with a particle size of less than about 400 nm.

90. The method of claim 89 wherein said drug substance is a steroid such as danazol or Steroid A or an antiviral agent.

91. The method of claim 89 wherein said surface modifier comprises gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene caster oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, and polyvinylpyrrolidone.

92. The method of claim 89 wherein said surface modifier comprises polyvinylpyrrolidone, an ethylene oxide-propylene oxide block copolymer, lecithin, an alkyl aryl polyether sulfonate, gum acacia, sodium dodecylsulfate, and a dioctylester of sodium sulfosuccinic acid.

93. The method of claim 89 wherein said array defines a lattice.

94. The method of claim 93 wherein the interstices among said filtering particles define the particle size of said drug substance.

95. A method of making drug formulations using a device comprising:
at least one inlet port and at least one exit port for said drug formulations;
a flow channel and reaction channel in communication with said ports; and
a filter zone within said reaction channel, with said filter zone comprising nanotubes arranged in parallel with the flow of fluid through said filter zone with the diameters of said nanotubes defining the drug formulation particle size;
said method comprising:
adding a drug substance and surface modifier through said inlet ports; and
cycling said drug substance and surface modifier through said reaction channel to provide drug formulations with a particle size of less than about 400 nm.

96. The method of claim 95 wherein said drug substance is a steroid such as danazol or Steroid A or an antiviral agent.

97. The method of claim 95 wherein said surface modifier comprises gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene caster oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, and polyvinylpyrrolidone.

98. The method of claim 95 wherein said surface modifier comprises polyvinylpyrrolidone, an ethylene oxide-propylene oxide block copolymer, lecithin, an alkyl aryl polyether sulfonate, gum acacia, sodium dodecylsulfate, and a dioctylester of sodium sulfosuccinic acid.

99. A method of making drug formulations using a device comprising:
at least one inlet port and at least one exit port for said drug formulations;
a flow channel and reaction channel in communication with said ports; and
a filter zone within said reaction channel, with said filter zone comprising nanopores etched into a substrate with the diameters of said nanopores defining the particle size;
said method comprising:
adding a drug substance and surface modifier through said inlet ports; and
cycling said drug substance and surface modifier through said reaction channel to provide drug formulations with a particle size of less than about 400 nm.

100. The method of claim 99 wherein said drug substance is a steroid such as danazol or Steroid A or an antiviral agent.

101. The method of claim 99 wherein said surface modifier comprises gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene caster oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, and polyvinylpyrrolidone.

102. The method of claim 99 wherein said surface modifier comprises polyvinylpyrrolidone, an ethylene oxide-propylene oxide block copolymer, lecithin, an alkyl aryl polyether sulfonate, gum acacia, sodium dodecylsulfate, and a dioctylester of sodium sulfosuccinic acid.
